(19) United States
(12) Patent Application Publication
(10) Pub. No.: US 2005/0260157 A1
(43) Pub. Date: Nov. 24, 2005

(54) Method and agent for treating vulnerable plaque

(75) Inventors: Patrice Tremble, Santa Rosa (CA); Wenda Carlyle, Silverado, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/531,851

(22) PCT Filed: Sep. 16, 2003
(86) PCT No.: PCT/US03/29062

§ 371 Date: Apr. 18, 2005

(51) Int. Cl. A61K 48/00; C12N 15/86

(52) U.S. Cl. 424/93.2; 514/44; 435/456

(57) ABSTRACT

A method and gene therapy agent for treating a vulnerable plaque associated with a blood vessel of a patient is disclosed. The method includes providing at least one gene therapy agent encoding at least one protein. The gene therapy agent is administered to a target cell population. The protein is expressed within the patient from a portion of the target cell population. The vulnerable plaque is modified as a result of the protein expression. The gene therapy agent includes at least one polynucleic acid encoding at least one protein. Administration of the gene therapy agent to a target cell population results in expression of the protein capable of modifying the vulnerable plaque.

### TECHNICAL FIELD OF THE INVENTION

The present invention relates generally to the field of vascular therapies. More particularly, the invention relates to a method and agent for treating a vulnerable plaque associated with a blood vessel of a patient.

### BACKGROUND OF THE INVENTION

Heart disease, specifically coronary artery disease (CAD), is a major cause of death, disability, and healthcare expense. Until recently, most heart disease was considered primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to heart muscles, causing a heart attack. Alternatively, the clot may break off and lodge in another organ vessel such as the brain resulting in a thrombotic stroke.

Within the past decade, evidence has emerged expanding the paradigm of atherosclerosis, coronary artery disease, and heart attacks. While the build up of hard plaque may produce angina and severe ischemia in the coronary arteries, new clinical data now suggests that the rupture of sometimes non-occlusive, vulnerable plaques causes the vast majority of heart attacks. The rate is estimated as high as 60-80 percent. In many instances vulnerable plaques do not impinge on the vessel lumen, rather, much like an abscess they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

The majority of vulnerable plaques include a lipid pool, necrotic smooth muscle (endothelial) cells, and a dense infiltrate of macrophages contained by a thin fibrous cap, some of which are only two micrometers thick or less. The lipid pool is believed to be formed because of a pathological process involving low density lipoprotein (LDL), macrophages, and the inflammatory process. The macrophages oxidize the LDL producing foam cells. The macrophages, foam cells, and associated endothelial cells release various substances, such as tumor necrosis factor, tissue factor, and matrix proteinases. These substances can result in generalized cell necrosis and apoptosis, pro-coagulation, and weakening of the fibrous cap. The inflammation process may weaken the fibrous cap to the extent that sufficient mechanical stress, such as that produced by increased blood pressure, may result in rupture. The lipid core and other contents of the vulnerable plaque (emboli) may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

The clear correlation between elevated serum cholesterol levels and the development of CAD is well established from numerous epidemiological and longitudinal studies. Lipoproteins play a major role in plasma cholesterol transport and their levels are commonly measured to determine risk for CAD. Unlike the other three major circulating lipoproteins, the high density lipoprotein (HDL) is primarily involved in the removal of cholesterol from peripheral tissues. HDL transports cholesterol back to the liver or to other lipoproteins by a process known as reverse cholesterol transport. The “protective” role of HDL has been confirmed in numerous in vitro and in vivo studies (Miller et al., 1977, Lancet 965-968; Whayne et al., 1981, Atherosclerosis 39:411-419; Badimon et al., 1992, Circulation 86:Suppl. III:86-94; and Koizumi et al., 1988, J. Lipid Res. 29:1405-1415). These studies have shown that elevated levels of LDL are clearly associated with CAD (i.e., presumably through a role in vulnerable plaque formation) whereas high HDL levels appear to confer cardiovascular protection.

Apolipoprotein A1 (Apo-A1) is the major component of the HDL particle and is thought to play an important role in HDL protection against CAD. Indeed, high plasma levels of Apo-A1 are associated with reduced risk of CAD and presence of coronary lesions (Gordon et al., 1989, N. Eng. J. Med. 321:1311-1316; Gordon et al., 1989, Circulation 79:8-15; and Rubin et al., 1991, Nature 353:265-267). Apo-A1 has been sequenced and comprises a single polypeptide chain of 243 amino acids (Brewer et al., 1978, Biochem. Biophys. Res. Commun. 80:623-630). The major functioning domain of the Apo-A1 molecule is believed to be the 11 or 22 repeating amino acids putatively forming an amphipathic helical formation (Segrest et al., 1974, FEBS Lett. 38:247-253). This structure presumable provides the main biologic activities associated with Apo-A1 (e.g., lipid binding and lecithin cholesterol acyl-transferase (LCAT) activation).

The apolipoprotein variant A1-Milano (Apo-A1 Milano) is the first described molecular variant of Apo-A1 (Franceschini et al., 1980, J. Clin. Invest. 66:892-900). It is characterized by a substitution of arginine-173 with a cysteine residue (Weisgraber et al., 1983, J. Biol. Chem. 258:2508-2513). The mutant apolipoprotein is transmitted as an autosomal dominant trait and eight generations of carriers have been identified (Gualandri et al., 1984, Am. J. Hum. Genet. 37:1083-1097). The status of the Apo-AIM carrier individual is characterized by a remarkable reduction in HDL-cholesterol level. In spite of this, the affected subjects do not apparently show any increased risk of arterial disease; indeed, by examination of the genealogic tree it appears that these subjects are “protected” from atherosclerosis.

The mechanism of the possible protective effect of Apo AI-M in the carriers seems to be linked to a modification in the structure of the mutant apolipoprotein, with the loss of one alpha-helix and an increased exposure of hydrophobic residues (Francheschini et al., 1985, J. Biol. Chem. 260:1632-1635). The loss of the tight structure of the multiple alpha-helices leads to an increased flexibility of the molecule, which associates more readily with lipids, compared to normal AI. Moreover, apolipoprotein/lipid complexes are more susceptible to denaturation, thus suggesting that lipid delivery is also improved in the case of the mutant.

Strategies have been developed for treating patients at risk for CAD with lipid lowering drugs and Apo-A1 Milano preparations. The lipid lowering drugs (e.g., bile-acid binding resins, statins, niacin and nicotinic acid, fibrates, and oral estrogens) each has its own drawbacks and limitations in terms of efficacy, side-effects, and qualifying patient population. Although advances have been made in the large-scale production of Apo-A1Milano for cardiovascular treatments, problems remain in its therapeutic use. For example, systemic administration of Apo-A1Milano may require repeated treatments over a long time period to achieve desirable results. In addition, patients successfully treated with Apo-A1Milano may not benefit from the therapy for long periods of time. As such, it would be desirable to provide an Apo-A1Milano treatment strategy that overcomes the disadvantages associated with the prior art.

Gene therapy science has developed in recent years and provides a potential treatment for many disorders. In gene therapy, somatic cells are modified in or ex vivo to express a gene corresponding to a therapeutically or diagnostically useful protein. The genetic information necessary to encode and express the protein is transferred into the cells by a number of techniques including injection, direct uptake, receptor-mediated uptake, electroporation, precipitation, and others. In vivo gene therapy typically involves the direct transfer of genetic material into a target cell group within a patient's body. Injection, direct uptake, receptor-mediated uptake, intravenous administration, and ingestion are generally used for this type of therapy. Alternatively, ex vivo therapy may involve removing a group of cells from the patient (e.g., “harvesting”) and transferring the genetic information in vitro followed by re-introduction of the modified cells back into the patient. The ex vivo therapy may also involve transferring the information to a variety of donor cells.

One area of gene therapy research relates to the circulatory system. Researchers have transferred genetic material to vascular tissues, including smooth muscle and endothelial cells. Engineered cells are capable of secreting the transferred protein for a significant period of time. For example, human adenosine deaminase was expressed in vivo by rat vascular smooth muscle cells for over six months (Lynch et al., 1993, Proc. Natl. Acad. Sci. USA 89:113842). Strategies are being developed to further prolong the stable expression of proteins by the transduced cells. Although gene therapy holds promise for the treatment of numerous vascular tissue disorders, effective therapies specific for vulnerable plaque lesions are still lacking. What is needed, therefore, is a vulnerable plaque treatment strategy that would take advantage of the benefits associated with gene therapy.

Accordingly, it would be desirable to provide a strategy for treating vulnerable plaque that would overcome the aforementioned and other disadvantages.

### SUMMARY OF THE INVENTION

One aspect according to the invention provides a method of treating a vulnerable plaque associated with a blood vessel of a patient. The method includes providing at least one gene therapy agent encoding at least one protein. The gene therapy agent is administered to a target cell population. The protein is expressed within the patient from a portion of the target cell population. The vulnerable plaque is modified as a result of the protein expression.

The gene therapy agent may include a polynucleic acid such as deoxyribonucleic acid and ribonucleic add. The gene therapy agent may include a vector such as a plasmid, retrovirus vectors, adenovirus vectors, Herpes Simplex vectors, Semliki Forest Virus vectors, and Sindbis virus vectors. The gene therapy agent may be administered by injection, direct uptake, receptor-mediated uptake, intravenous administration, ingestion, electroporation, and precipitation.

The gene therapy agent may be administered in vivo the patient. The in vivo gene therapy may be administered with a balloon catheter device, by stenting the blood vessel adjacent the vulnerable plaque, and/or by interstitial administration.

The gene therapy agent may be administered ex vivo the patient. The ex vivo gene therapy may include harvesting the cell population from the patient, selecting for the portion of target cells capable of expressing the protein subsequent the administration of the gene therapy agent, and administering the selected cells into the patient. The selected cells may be reintroduced into a pericardial space of the patient. Alternatively, the therapeutic protein may be introduced into allogenic cells using any acceptable technique, including plasmid delivery, any of a variety of vector delivery techniques or any other suitable technique, where the transfected cells are thereafter introduced into the patient using an immunoisolation device.

The protein may be a collagen isoform or an A1 apolipoprotein isoform, such as a mutant Milano isoform. The target cell population may be muscle cells, vascular cells, hepatic cells, harvested patient cells, and/or donor cells. Expressing the protein may include secreting the protein into a bloodstream, localized expression adjacent the vulnerable plaque, and/or modulating expression level with an expression cassette. Modifying the vulnerable plaque may include fibrous cap reinforcement, reduction of lipid pool size, modifying a lipid pool constitution, modifying an inflammation response, preventing vulnerable plaque formation, and/or preventing vulnerable plaque enlargement.

Another aspect according to the invention provides a gene therapy agent for treating a vulnerable plaque associated with a blood vessel of a patient. The gene therapy agent includes at least one polynucleic acid encoding at least one protein. Administration of the gene therapy agent to a target cell population results in expression of the protein capable of modifying the vulnerable plaque.

The polynucleic acid may be deoxyribonucleic acid and/or ribonucleic acid. The protein may be a collagen isoform or an A1 apolipoprotein isoform such as a mutant Milano isoform. The gene therapy agent may further include a vector operably attached to the polynucleic acid. The vector may be a plasmid, retrovirus vector, adenovirus vector, Herpes Simplex vector, Semliki Forest Virus vector, and Sindbis virus vector. The gene therapy agent may further include a liposome sheathing the gene therapy agent and/or an expression cassette encoded in the polynucleic acid.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a flow chart of a method of treating a vulnerable plaque associated with a blood vessel of a patient, in accordance with one embodiment of the present invention;

FIG. 2 shows a representative gene therapy vector according to one embodiment of the present invention;

FIGS. 3A and 3B show the 729 base DNA and 243 amino acid sequences of a representative apolipoprotein-A1 isoform. The Milano isoform is produced by a C-to-T transition resulting in expression of a unique cysteine residue (Cys173), the mutated locations shown by underline;

FIG. 4 is a schematic view of a balloon catheter used to administer a gene therapy agent according to one embodiment of the present invention; and

FIG. 5 is a schematic view of a balloon catheter used to administer a gene therapy agent according to another embodiment of the present invention.

### DESCRIPTION OF THE PRESENTLY PREFERRED EMBODIMENTS

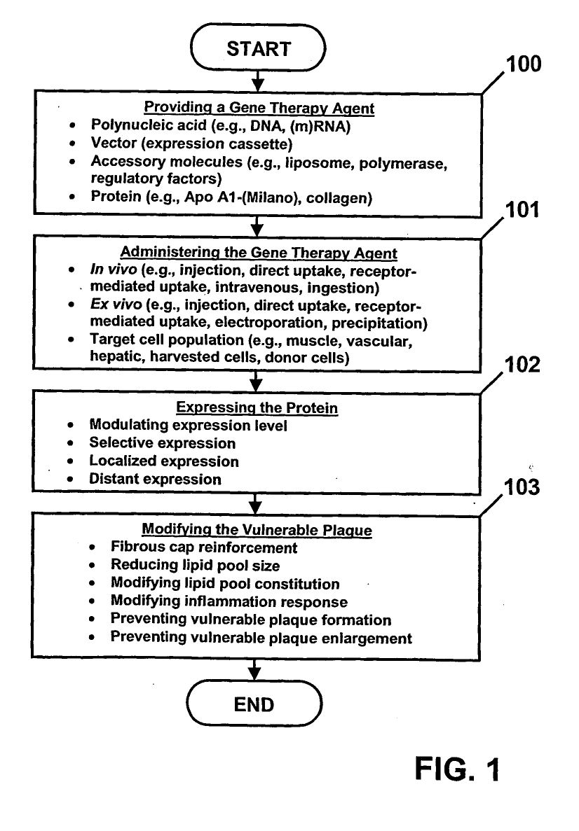

Referring to the drawings, wherein like reference numerals refer to like elements, FIG. 1 is a flow chart of a method of treating a vulnerable plaque associated with a blood vessel of a patient, in accordance with one embodiment of the present invention. A vulnerable plaque is distinguishable from other types of plaque, including hard plaques, by the presence of a fibrous cap. The vulnerable plaque fibrous cap retains a pool of lipids and other contents, which may be released into the blood vessel upon rupture. The released contents may form emboli that can lodge in a blood vessel thereby posing a risk to the patient. Vulnerable plaques, unlike hard plaques, are generally non-occlusive and as such, may not produce angina. The following description pertains to treatment of these vulnerable plaques.

Those skilled in the art will recognize that although the present invention is described primarily in the context of treating a vulnerable plaque while using specific gene therapy agents, the inventors contemplate a broader method of application. Any number of treatment agents capable of performing the prescribed function(s) may be compatible with the present invention. Furthermore, the treatment of the vulnerable plaque is not limited to the described methodology. Numerous modifications, substitutions, and variations may be made to the method and gene therapy agents while providing effective vulnerable plaque treatment consistent with the present invention.

The term “target cell population,” as used herein, includes cells, tissues, and/or organs. The term “gene therapy agent”, as used herein, includes polynucleic add constructs which are single, double or triplex stranded, linear or circular, that are expressible or non-expressible constructs which can either encode for and express a functional protein, or fragment thereof, or interfere with the normal expression of a target gene, gene transfer and/or expression vectors. The term “gene therapy agent”, may further include accessory molecules such as chemical drugs, protein drugs, nucleic acid drugs, combination chemical/protein/nucleic acid drugs, liposomes, and the like, which typically have a therapeutic effect on vulnerable plaque and/or facilitate function of the gene therapy agent.

As shown in FIG. 1, vulnerable plaque treatment may begin by providing a gene therapy agent (step 100). The composition of the gene therapy agent may vary depending on the treatment strategy. In one embodiment, the gene therapy agent may include a polynucleic acid polymer of ribonucleic acid (RNA), which encodes one or more proteins (e.g., polypeptides). Specifically, the agent may include messenger RNA (mRNA) for directing transitory protein expression. Use of mRNA typically provides protein expression for only about one day and does not require nuclear penetration. As such, there is typically no genetic liability (e.g., transformation, insertion, mutation, etc.).

In some situations, however, a more prolonged effect may be desired without incorporation of the exogenous polynucleic acid into the host cell genome. In order to provide such an effect, another embodiment of the invention provides a gene therapy agent including a polymer of deoxyribonucleic acid (DNA), which encodes one or more proteins. Non-replicating DNA sequences can be introduced into a target cell population to provide production of a desired protein for periods of about up to six months and without integration of the DNA sequence into the cell genome.

An even more prolonged effect may be achieved by introducing the DNA into the target cell by means of a vector having the DNA sequence inserted therein. Examples of vectors that may be adapted for use with the present invention include, but are not limited to, a plasmid, retrovirus vectors, adenovirus vectors, Herpes Simplex vectors, Semliki Forest Virus vectors, and Sindbis virus vectors. Such vectors are well known to those skilled in the art and may be used to provide efficient introduction of a polynucleic acid into the target cell.

The vector may include an expression cassette for modulating protein expression level. In the present description, the term “expression cassette” refers to one or more polynucleotide sequences positioned within the vector for modulating the replication, transcription, translation, integration, secretion, and/or degradation of the vector, resulting mRNA, and/or resulting protein. In one embodiment, the expression cassette may include a replicator sequence for enhancing replication of the vector and ultimately increasing protein expression level. Other examples of vectors with an expression cassette that may be adapted for use with the present invention include plasmid pBR322, with replicator pMB1, or plasmid pMK16, with replicator ColE1.

In another embodiment, the expression cassette may include a cell-specific promoter that permits protein expression only in predetermined cells. In another embodiment, the vector may encode a polymerase for transcribing portions of the vector wherein the polymerase may bind to specific recognition sites on the expression cassette. The encoded polymerase may either recognize a generic promoter region (i.e., common to many genes) or a unique promoter region (i.e., specific to the vector).

In another embodiment, the expression cassette may include any number of enhancer or repressors, which are known in the art for modulating transcription and, ultimately, protein expression level of a given polynucleotide sequence. In another embodiment, the expression cassette may include encoded regions for binding of translation enhancer and/or repressor proteins to any transcribed mRNA sequence. Protein translation level may be increased or decreased by the binding of the enhancer or repressor proteins, respectively, to the mRNA. In another embodiment, the expression cassette may include sequence code that controls integration into the target cell genome, which may prolong protein expression. In another embodiment, the expression cassette may include sequence code that directs the secretion of the protein.

Those skilled in the art will recognize that a myriad of strategies exist for modulating protein expression. Numerous such strategies may be adapted for use with the present invention. For example, protein expression level may be modulated by changing mRNA stability through incorporation of AU-rich sequences in its 3′ untranslated region (UTR). Such sequences have been shown to accelerate mRNA degradation by stimulating the removal of the poly-A tail. As another example, recognition sites may be provided in the 3′ UTR for specific endonucleases to cleave the mRNA. Such degradation promoting sequences may be encoded in the polynucleic acid expression cassette of the gene therapy agent to modulate (e.g., decrease) protein expression. Alternatively, various strategies for increasing protein expression and enhancing mRNA stability are known in the art and may be adapted for use with the present invention.

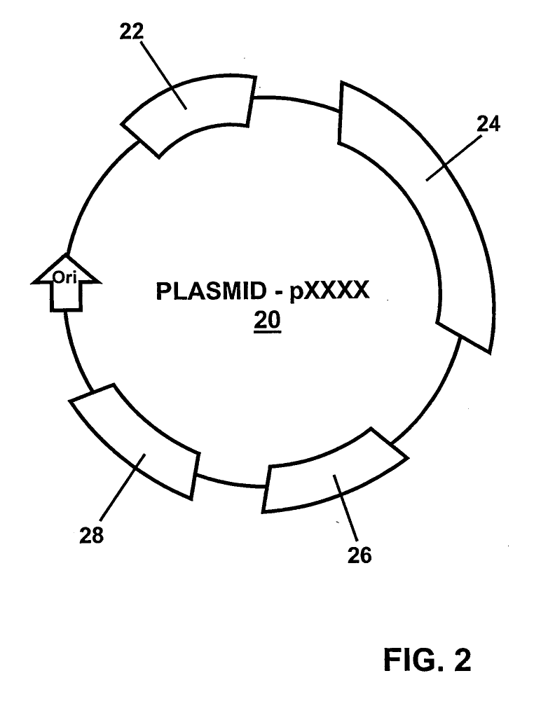

FIG. 2 shows a representative gene therapy vector 20 according to one embodiment of the present invention. Vector 20, which in this case is a double-stranded DNA plasmid, includes an expression cassette 22, encoded protein 24, and two selection genes 26, 28. Selection genes may be provided to aid gene therapy administration in terms of selecting those cells that include vector and are capable of expressing the protein. Those skilled in the art will recognize that the configuration, constitution, and number of genes may vary greatly within a given vector. The representative vector 20 is provided merely as one example. The design of the vector generally will vary depending on the method of gene therapy administration.

In one embodiment, an immediate and long lived gene expression may be achieved by utilizing a gene therapy agent including a liposomal preparation with both DNA and an RNA polymerase, such as the phage polymerases T7, T3, and SP6. The liposome sheath or particle may also include an initial source of the appropriate RNA polymerase, by either including the actual enzyme itself, or alternatively, an mRNA coding for that enzyme. When the liposome is introduced into the target cell, it delivers the DNA and the initial source of RNA polymerase. The RNA polymerase, recognizing the promoters on the introduced DNA, transcribes both genes, resulting in translation products comprising more RNA polymerase and the desired protein. Production of these materials continues until the introduced DNA, which may be in the form of a plasmid, is degraded. In this manner, production of the desired protein in vivo may be achieved in a few hours and be extended for one month or more.

The expression of the gene therapy agent encoded protein of the present invention typically has a therapeutic effect on vulnerable plaque. In one embodiment, the protein may be a collagen isoform. Numerous isoforms of collagen are known in the art and may have beneficial effects on the treatment of vulnerable plaque. For example, localized collagen expression may reinforce or strengthen the fibrous cap thereby reducing chance of vulnerable plaque rupture.

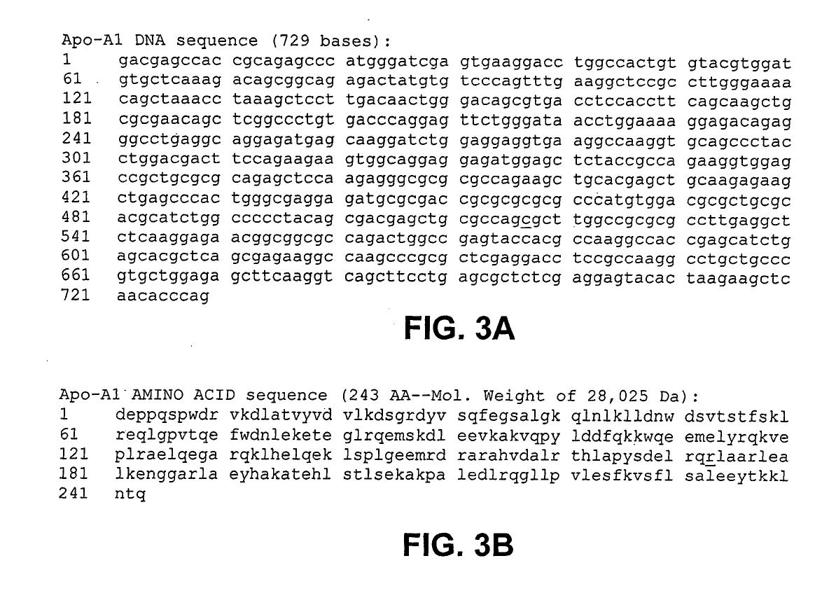

In another embodiment, the encoded protein may be an A1 apolipoprotein isoform wherein the protein may also include the Apo-A1 Milano mutation. Exemplary DNA and protein sequences of human mature Apo-A1 are shown in FIGS. 3A and 3B, respectively. The mature protein consists of 243 amino acids, as shown, whereby it may be formed by proteolitic cleavage of 24 amino acids during its formation. The Milano isoform, or Apo-A1 M, includes a single base C-to-T transition thereby changing the codon of CGC to TGC. The altered codon produces an amino acid change of Arg173 to Cys173, putatively allowing dimerisation of the molecule.

In yet another embodiment, any number of proteins that may have a beneficial effect on vulnerable plaque treatment may be used with the present invention. It should be noted that that the specific protein examples described herein may vary (i.e., using an isoform of another species or of another predetermined design). In addition, the inventors contemplate that numerous other protein may be adapted for use with the present invention and are not limited to the examples discussed herein.

After an appropriate gene therapy agent is provided, it is administered to a target cell population (step 101). The method of administration generally depends on whether the target cell population will be treated in vivo or ex vivo. With in vivo gene therapy, the gene therapy agent is administered to a patient using a variety of methods. The gene therapy agent transforms a target cell population within the patient followed by protein expression. With ex vivo gene therapy, the gene therapy agent is administered to a target cell population outside of the patient's body. The cells expressing the gene therapy protein(s) may then be administered to the patient, e.g., through the use of one or more an immunoisolation devices. In vivo gene therapy generally requires fewer steps; however, ex vivo therapy can provide greater flexibility. It should be noted that, with either method, more than one gene therapy agent may be administered to provide vulnerable plaque treatment. In addition, each gene therapy agent may express more than one mRNA and/or protein and, optionally, include one or more accessory drug or cofactor (e.g., therapeutic agent, enzyme, protein, lipid, nucleic acid, etc.).

With in vivo gene therapy, the agent may be administered to the patient by numerous methods including, but not limited to injection, direct uptake, receptor-mediated uptake, intravenous administration, and ingestion, all of which are known in the art. In one embodiment, the gene therapy agent may be injected (e.g., by needle or by sheer force through the cell membrane) into target cell population of a given tissue. In another embodiment, the agent may be administered in proximity to the target cell population allowing eventual uptake through direct tissue contact (e.g., direct uptake). Gene therapy agents associated with liposomes and naked nucleic acids may be suited for direct uptake administration. In another embodiment, the agent may be administered by receptor-mediated uptake by the target cell population. Gene therapy agents associated with antibody coated liposomes or particles may be suited for receptor-mediated uptake. Furthermore, the antibodies may be used to target the liposomes/particle to a specific cell target population, such as a certain tissue or organ. In another embodiment, the gene therapy agent may be administered intravenously to the patient. In another embodiment, the gene therapy agent may be ingested (e.g., orally or nasally) by the patient. Gene therapy agents associated with viral vectors may be suited for ingestion. Those skilled in the art will recognize that the gene therapy agent may be administered to the patient by a variety of methods, including methods not here described.

The gene therapy agent may be delivered directly into the tissue mass and/or into the interstitial space of tissues of the patient, including those of muscle, skin, brain, lung, liver, spleen, bone marrow, thymus, heart, lymph, blood, blood vessel, bone, cartilage, pancreas, kidney, gall bladder, stomach, intestine, testis, ovary, uterus, rectum, nervous system, eye, gland, and connective tissue. The interstitial space of the tissues includes the periadventitial, intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. The agent may be preferably administered to and protein expressed in persistent, non-dividing cells which are differentiated, although delivery and expression may be achieved in non-differentiated or less completely differentiated cells, such as, for example, stem cells of blood or skin fibroblasts.

Muscle tissue provides an attractive gene therapy target as the cells are typically competent in the ability to take up and express polynucleotides. This ability may be due to the singular tissue architecture of muscle, comprising multinucleated cells, sarcoplasmic reticulum, and transverse tubular system. Polynucleotides may enter the muscle through the transverse tubular system, which contains extracellular fluid and extends deep into the muscle cell. It is also possible that the polynucleotides enter damaged muscle cells which then recover. Muscle is also advantageously used as a site for the delivery and expression of polynucleotides in a number of therapeutic applications because humans have a proportionately large muscle mass which is conveniently accessed by direct injection through the skin. For this reason, a comparatively large dose of a gene therapy agent can be deposited in muscle by multiple injections, and repetitive injections, to extend therapy over long periods of time. In addition, the procedure may be easily performed and may be carried out safely and without special skill or devices.

Vascular tissue may be advantageously chosen as the gene therapy target as the expressed protein may have a direct therapeutic role on the vulnerable plaque. As such, the gene therapy protein may be expressed within the cells directly involved in and/or adjacent to the vulnerable plaque. In one embodiment, the gene therapy agent may be administered in vivo with a balloon catheter device, which may provide localized and minimally invasive gene therapy administration. Such devices are typically positioned within a blood vessel adjacent the vulnerable plaque prior to gene therapy agent delivery. The balloon catheter may include features that facilitate the administration of the gene therapy agent. For example, the balloon catheter may include one or more deployable needles for injection of the gene therapy agent into the vascular tissue. The catheter may include dual balloon portions positioned upstream or downstream of the vulnerable plaque thereby allowing stoppage of blood flow that would interfere with intravascular gene therapy agent administration. Alternatively, the blood flow of the target vessel may be stopped by ligation. The catheter may include an expandable stent deployed on the balloon. The stent may further include a drug coating that, for example, includes the gene therapy agent. Such a drug coated stent would provide a prolonged and localized administration of the gene therapy and/or other therapeutic agents.

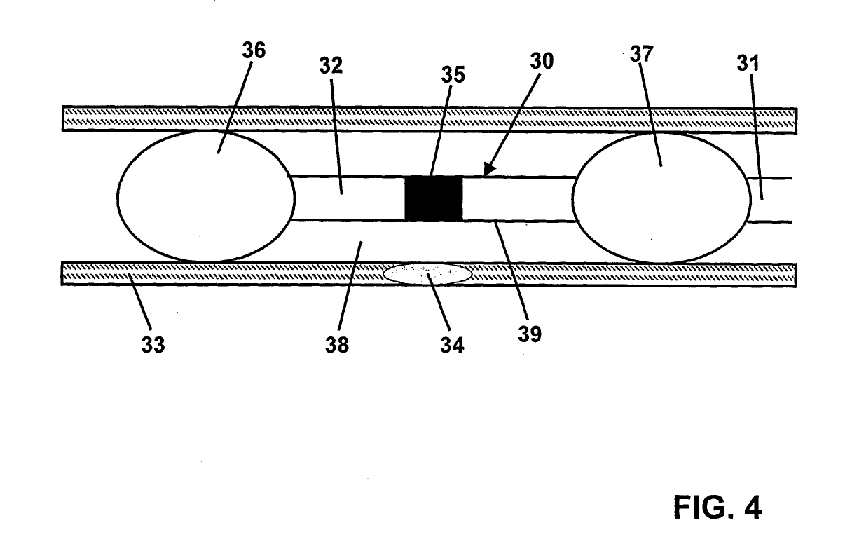

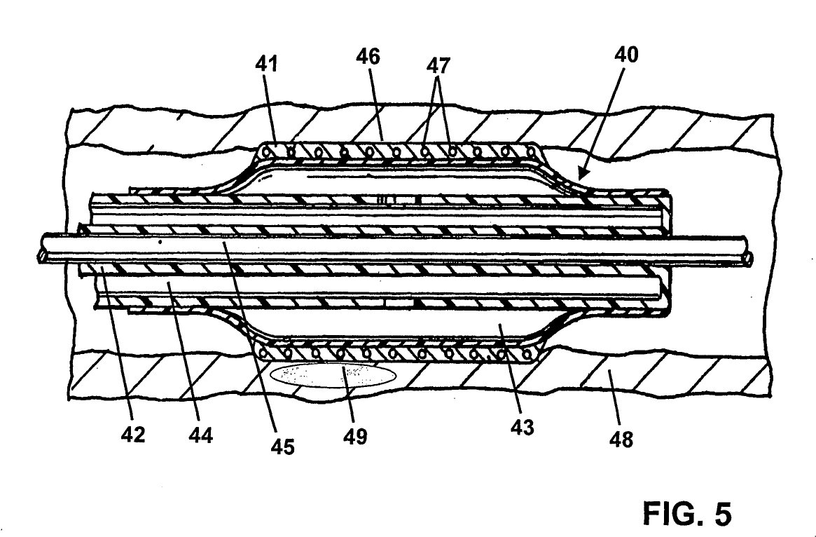

Numerous balloon catheter devices for administering therapeutic agents and/or gene therapy agents are known in the art and may be adapted for use with the present invention. By way of example, the device 30 shown in FIG. 4 and device 40 shown in FIG. 5 are balloon catheters that may be used to deliver a gene therapy agent according to the present invention. In one embodiment, device 30 includes an elongated hollow flexible tubular member 31 attached to an operating head 32, which is shown inserted within a blood vessel 33 including a vulnerable plaque 34. The operating head 32 contains a particle bombardment device 35 disposed therein for discharging gene therapy agent-coated particles through a discharge port (not shown).

The particles may be manufactured from a chemically inert substance such as gold or diamond (either synthetic or natural) and may be coated with the gene therapy agent of the present invention. The particles are accelerated within the bombardment device 35 out of the discharge port toward a predetermined target tissue, which in this case is the blood vessel 33 wall. The particles carry enough momentum to penetrate the target tissue thereby allowing intracellular delivery of the gene therapy agent.

Device 30 may also include proximal and distal balloons 36, 37, which are shown in an inflated state, girthing the tubular member 31 on either side of the operating head 32. A balloon inflation line (not shown) extends through the device 30 providing inflation pressures to the balloons 36, 37. Inflation of the balloons 36, 37 against the blood vessel 33 walls serves to isolate a space 38 there between through which the gene therapy agent-coated particles may be discharged. The particles may exit through the operating head 32 and/or a side wall 39 of the device 30 toward the target cells. The device 30 may also include suction and gas injection ports in communication with suction and gas injection lines (not shown) thereby allowing any body fluids in the space 38 isolated by the proximal and distal balloons 36, 37 to be displaced by gas. Displacement of fluid from the space 38 prior to gene therapy administration may better expose the targeted cells and minimize frictional drag on the accelerated particles. Device 30 may be analogous to that described in U.S. Pat. No. 5,836,905 to Lemelson et al., which is incorporated by reference herein.

In another embodiment, as shown in FIG. 5, device 40 includes a balloon 41 disposed adjacent one end of an elongated hollow flexible tubular member 42. Balloon 41, which is shown in an inflated state, defines an interior chamber 43 in communication with a lumen 44 formed within the tubular member 42 to facilitate balloon 41 inflation and deflation. An inner lumen 45 is sized to permit a guidewire to pass therethrough. Balloon 41 includes an outer peripheral surface 46, on which a plurality of microencapsulated spheres 47 are impregnated in a coating material, which may be, but is not limited to, a hydrophilic material. The microencapsulated spheres 47, which are immersed in the coating material, include the gene therapy agent of the present invention. The microencapsulated spheres 47 may be extruded in the balloon 41 wall during the manufacturing process. The microencapsulated spheres 47 may be manufactured from a biologically inert material, such as a polymeric material, and are sized (e.g., on the order of 5 microns) and configured to rupture upon application of a predetermined pressure caused by inflating the balloon 41. The microencapsulated spheres 47 are fabricated with a quantity of the gene therapy agent in accordance with known techniques. The spheres 47 become embedded in a vessel wall 48 when an initial pressure is communicated to the balloon 41, and thereafter rupture upon further inflation of the balloon 41 thereby administering the gene therapy agent to the patient. Device 40 may be analogous to that described in U.S. Pat. No. 6,129,705 to Grantz, which is incorporated by reference herein.

Those skilled in the art will recognize that numerous other balloon catheter devices may be adapted for use with the present invention, including a perforated balloon catheter, the pulse voltage needle cannula device described in U.S. Pat. No. 5,702,384 to Umeyama et al., the balloon catheter with channel forming means described in U.S. Pat. No. 5,997,525 to March et al. as well as other injection-based catheter devices, such as that shown in U.S. Pat. No. 5,112,305 to Barath or U.S. Pat. No. 5,354,279 to Hofling, each of which are incorporated both by reference herein. It should be noted that these balloon catheter delivery devices may also be adapted to administer the gene therapy agent to tissues other than vascular tissue.

The in vivo gene therapy may include stenting the blood vessel adjacent the vulnerable plaque. Numerous stent devices are known in the art and may be adapted for use with the present invention. The stent typically maintains the inner diameter size of the vessel thereby reducing a tendency of stentosis. Preferably, the stent includes a drug coating for delivering the gene therapy agents and possibly other therapeutic agents. As such, a prolonged local administration of the gene therapy agent may be provided. The stent may be self-expanding or balloon-expandable and deployed by methods known in the art. In one embodiment, the stent may include a polymer film drug coating analogous to that described in U.S. Pat. No. 5,700,286 to Tartaglia et al., which is incorporated by reference herein. In another embodiment, the drug coating may be prepared by a method analogous to that described in U.S. Pat. No. 6,358,556 to Ding et al., which is incorporated by reference herein.

It should be noted that numerous devices known in the art, other than balloon catheter devices and stents, may be adapted for in vivo gene therapy administration of the present invention. The minimally invasive needle catheter device described in U.S. Pat. No. 6,322,536 to Rosengart et al. is an example of one such device. It should also be noted that tissues other than those of muscle or blood vessel, and having a less efficient expression of injected polynucleotides or a less proximate relationship to the vulnerable plaque, may nonetheless be advantageously used as administration sites to produce therapeutic results. In this application, and in many others, such as those in which an enzyme or hormone is the gene product, it is not necessary to achieve high levels of protein expression in order to effect a valuable therapeutic result. Alternatively, it is possible that a certain tissue may be chosen on its ability to correctly process and secrete a “product” capable of having a therapeutic affect on vulnerable plaque. For example, the liver may be used because of its ability to secrete mature HDL particles, which contain the A1 apolipoprotein. Secreted HDL particles may enter the circulation thereby providing a distal therapeutic effect on the vulnerable plaque.

With ex vivo gene therapy, the gene therapy agent is administered to a target cell population outside of the patient's body. The cells are may be harvested from the patient or obtained from a donor source. The gene therapy agent may be administered to the harvested and donor cells in vitro. Alternatively, the gene therapy agent may be administered to the donor cells in another organism. Those skilled in the art will recognize that the nature and source of the harvested and donor cells may vary. For example, the donor cells may originate from another species and may be engineered (i.e., to reduce immunoreactivity), of cloned or stem-line origin, and the like.

After the gene therapy agent is administered, the cells expressing the gene therapy protein (e.g., the “transduced” cells) may then be administered to the patient. The gene therapy agent may be administered to the target cell population by numerous methods including, but not limited to, injection, direct uptake, receptor-mediated uptake, electroporation, and precipitation. In one embodiment, the gene therapy agent may be microinjected into a target cell, and the target cell may divide to form a target cell population. In another embodiment, the agent may be administered to the cells by direct uptake (i.e., through incubation of the gene therapy agent with the target cells). Gene therapy agents associated with liposomes (i.e., lipofection) and naked nucleic acids may be suited for direct uptake administration.

In another embodiment, the agent may be administered by receptor-mediated uptake by the target cell population. Gene therapy agents associated with antibody coated liposomes or particles may be suited for receptor-mediated uptake. In another embodiment, the gene therapy agent may be administered by electroporation (i.e., subjecting the target cell population to short bursts of electro shock). In another embodiment, the gene therapy may be administered by a precipitation protocol known in the art. Those skilled in the art will recognize that the gene therapy agent may be administered to the target cell population by a variety of methods, including methods not here described.

The administration of the gene therapy agent varies in efficiency, and is typically inefficient. As such, the protein may be expressed from a small proportion of the target cell population. It is sometimes desirable in the case of ex vivo administration to select for the portion of cells expressing the protein (i.e., by including one or more selection markers) thereby increasing the “potency” of the administered cells. The transduced cells may be administered to the patient by a variety of methods including, but not limited to, injection and implantation. Numerous such methods are known in the art. In one embodiment, transduced target cells may be injected with a needle or cannula device into a desired tissue or location of the patient. More specifically, the selected cells may be (re)introduced into a pericardial space of the patient thereby providing localized gene therapy adjacent heart tissues. In another embodiment, a stent or vascular graft seeded with transduced endothelial cells may be implanted within a blood vessel adjacent the vulnerable plaque. Those skilled in the art will recognize that the transduced cells may be administered to the patient by a variety of methods, including methods not here described.

After the gene therapy agent has been administered to the target cell population and, optionally, the selected cells administered to the patient, the protein is expressed within the patient from a portion of the target cell population (step 102). The expression level and duration of the protein may be influenced and controlled by a variety of factors. For example, as previously discussed, sequences provided in the expression cassette may modulate replication, transcription, translation, integration, secretion, and/or degradation of the vector, resulting mRNA, and/or resulting protein. Furthermore, modulator proteins encoded in a gene therapy agent may control expression level and duration. Protein expression level and duration, however, need not be modulated through an encoded sequence, but may be achieved by providing other factors included with the gene therapy agent. Protein regulators, polymerases, and other cofactors, for instance, may be included within a liposome. Protein expression may also be cell-specific by, for example, including a cell-specific promoter in the expression cassette.

The gene therapy agent protein may be expressed in cells adjacent to or relatively distant from the vulnerable plaque. In one embodiment, the protein may be expressed is vascular tissue thereby providing a localized expression adjacent the vulnerable plaque. This may be advantageous for expression of, for example, a collagen protein thereby reinforcing the vulnerable plaque fibrous cap. In another embodiment, the protein may be expressed in a tissue distant from the vulnerable plaque. The protein(s) may then be secreted into the bloodstream thereby allowing a direct therapeutic effect on the vulnerable plaque. This may be advantageous for expression of, for example, Apo-A1 Milano. Distant tissue expression may be used because certain protein(s) may only be capable of expression, packaging, and/or secretion in a certain tissue, such as the liver. The distant tissue may also provide more favorable expression levels and/or feasibility of administration. For example, muscle tissue provides a large target mass as well as proximity to the skin to facilitate administration by injection.

The expressed protein may then have either a direct or indirect role in modifying vulnerable plaque (step 103). Vulnerable plaque modification includes any changes of therapeutic benefit in the treatment of vulnerable plaque. The changes include fibrous cap reinforcement (e.g., thickening the fibrous cap by collagen expression), reduction of lipid pool size (e.g., enhancing cholesterol transport by Apo-A1 Milano expression), modifying a lipid pool constitution (e.g., by expressing proteins or factors such as anticoagulants secreted into the lipid pool), modifying an inflammation response (e.g., by expressing anti-inflammatory proteins), preventing vulnerable plaque formation (e.g., by Apo-A1 Milano expression), and preventing vulnerable plaque enlargement (e.g., by Apo-A1 Milano expression). The gene therapy agent protein may be expressed on either a short or long-term basis and as a current treatment of vulnerable plaque or as a prophylactic. In one embodiment, Apo-A1 Milano, or another protein, may be expressed in a patient after the detection of vulnerable plaque as a current treatment. In another embodiment, Apo-A1 Milano, or another protein, may be expressed in a patient as a preventative treatment, especially ff vulnerable plaque is suspected. In either case, protein expression according to the present invention may be preferable to simple administration of the purified protein for vulnerable plaque treatment. Gene therapy offers the advantages of expression time and level modulation, tissue expressive targeting, and native protein packaging and secretion, all of which may not be provided by other methods.

While the embodiments of the invention disclosed herein are presently considered to be preferred, various changes and modifications may be made without departing from the spirit and scope of the invention. The gene therapy agents, devices and their method of use are not limited to any particular composition, design, type, or sequence. Moreover, the procedure step order and method of achieving the same may vary without limiting the utility of the invention. Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. A method of treating a vulnerable plaque associated with a blood vessel of a patient, the method comprising:
providing at least one gene therapy agent encoding at least one protein;
administering the gene therapy agent to a target cell population;
expressing the protein within the patient from a portion of the target cell population; and
modifying the vulnerable plaque as a result of the protein expression.

2. The method of claim 1 wherein the gene therapy agent comprises a polynucleic acid selected from a group consisting of deoxyribonucleic acid and ribonucleic acid.

3. The method of claim 1 wherein the gene therapy agent comprises a vector selected from a group consisting of a plasmid, retrovirus vectors, adenovirus vectors, Herpes Simplex vectors, Semliki Forest Virus vectors, and Sindbis virus vectors.

4. The method of claim 1 wherein the gene therapy agent administration comprises at least one technique selected from a group consisting of injection, direct uptake, receptor-mediated uptake, intravenous administration, ingestion, electroporation, and precipitation.

5. The method of claim 1 wherein the gene therapy agent is administered in vivo the patient.

6. The method of claim 5 wherein the in vivo gene therapy is administered with a balloon catheter device.

7. The method of claim 5 wherein the in vivo gene therapy comprises stenting the blood vessel adjacent the vulnerable plaque.

8. The method of claim 5 wherein the in vivo gene therapy is administered interstitially.

9. The method of claims 1 wherein the gene therapy agent is administered ex vivo the patient.

10. The method of claim 9 further comprising:
harvesting the cell population from the patient;
selecting for the portion of target cells capable of expressing the protein subsequent the administration of the gene therapy agent; and
administering the selected cells into the patient.

11. The method of claim 10 wherein the selected cells are reintroduced into a pericardial space of the patient.

12. The method of claim 1 wherein the protein is a collagen isoform.

13. The method of claim 1 wherein the protein is an A1 apolipoprotein isoform.

14. The method of claim 13 wherein the A1 apolipoprotein is a mutant Milano isoform.

15. The method of claim 1 wherein the target cell population comprises cells selected from a group consisting of muscle cells, vascular cells, hepatic cells, harvested patient cells, and donor cells.

16. The method of claim 1 wherein expressing the protein comprises secreting the protein into a bloodstream.

17. The method of claim 1 wherein expressing the protein comprises localized expression adjacent the vulnerable plaque.

18. The method of claim 1 wherein expressing the protein comprises modulating expression level with an expression cassette.

19. The method of claim 1 wherein modifying the vulnerable plaque comprises a modification selected from a group consisting of fibrous cap reinforcement, reduction of lipid pool size, modifying a lipid pool constitution, modifying an inflammation response, preventing vulnerable plaque formation, and preventing vulnerable plaque enlargement.

20. A gene therapy agent for treating a vulnerable plaque associated with a blood vessel of a patient, the gene therapy agent comprising:
at least one polynucleic acid encoding at least one protein wherein administration of the gene therapy agent to a target cell population results in expression of the protein capable of modifying the vulnerable plaque.

21. The gene therapy agent of claim 20 wherein the polynucleic acid selected from a group consisting of deoxyribonucleic acid and ribonucleic acid.

22. The gene therapy agent of claim 20 wherein the protein is a collagen isoform.

23. The gene therapy agent of claim 20 wherein the protein is an A1 isoform of an apolipoprotein.

24. The gene therapy agent of claim 23 wherein the A1 apolipoprotein is a mutant Milano isoform.

25. The gene therapy agent of claim 20 further comprising a vector operable attached to the polynucleic acid.

26. The gene therapy agent of claim 25 wherein the vector is selected from a group consisting of a plasmid, retrovirus vectors, adenovirus vectors, Herpes Simplex vectors, Semliki Forest Virus vectors, and Sindbis virus vectors.

27. The gene therapy agent of claim 20 further comprising a liposome sheathing the gene therapy agent.

28. The gene therapy agent of claim 20 further comprising an expression cassette encoded in the polynucleic acid.
